(19) United States
(12) Patent Application Publication
(10) Pub. No.: US 2022/0105063 A1
(43) Pub. Date: Apr. 7, 2022

(54) Composition for Treatment and / or Prevention of Infectious Hoof Diseases

(71) Applicant: Nutrition Sciences N.V., Drongen (BE)

(72) Inventors: Geert BRUGGEMAN, Brugge (BE); Roland BRUGGER, Ghent (BE)

(21) Appl. No.: 17/427,903

(22) PCT Filed: Feb. 10, 2020
(86) PCT No.: PCT/EP2020/053266

§ 371 Date: Aug. 2, 2021

(30) Foreign Application Priority Data
Feb. 8, 2019 (BE) 2019/5081

(51) Int. Cl. A61K 31/20; A61K 45/06; A61K 31/194; A61K 9/00

(52) U.S. Cl. CPC A61K 31/20; A61K 9/0017; A61K 31/194; A61K 45/06

(57) ABSTRACT

The current invention provides a composition for use in the topical treatment and/or prevention of infectious hoof diseases in an animal, wherein composition comprises a medium-chain fatty acid (MCFA) fraction comprising one or more MCFAs and/or derivatives thereof. In a second aspect, the invention provides a method for disinfecting the hoofs of an animal comprising the step of topically administering a composition comprising an MCFA fraction to said animal, wherein said MCFA fraction comprises one or more MCFAs and/or derivatives thereof. In a final aspect, the invention provides a disinfectant composition comprising an MCFA fraction and acarboxylic acid (CA) fraction, wherein said MCFA fraction comprises MCFAs and/or derivatives thereof, and said CA fraction comprises one or more carboxylic acids.

### TECHNICAL FIELD

The invention is directed to compositions for use in the treatment and/or prevention of infectious hoof diseases in an animal and methods for enhancing the zootechnical performance of animals using such compositions.

### BACKGROUND

Infectious diseases of the hoof, such as hairy hoof warts (papillomatous digital dermatitis), hoof rot (interdigital phlegmon), and stable hoof rot (interdigital dermatitis) are common in farm animals such as sheep, goats, horses, dairy cows and beef cattle.

Digital dermatitis is a disease that causes lameness in cattle. It is an infectious disease caused by multiple different bacteria and has been described throughout the world, mainly in high density housing and intensive production dairy systems.

Symptoms of digital dermatitis include decreased mobility, lifting of the affected leg, walking with a toe down posture, severe lameness and decreased milk production. These symptoms have a detrimental effect on animal welfare and also pose a serious economic burden on producers due to lower production, increased reproductive intervals and increased culling.

There is a need in farming for an improved treatment or prevention of infectious hoof diseases in calves, cattle and dairy cattle. A known approach to treat digital dermatitis includes the topical application of antibiotics. Other approaches focus on preventing the disease using foot baths with formalin, copper sulfate or thymol-based disinfectants. WO2008031087 describes a disinfectant composition comprising at least one C2-C14 carboxylic acid and at least one surfactant for the treatment and/or prevention of hoof diseases.

The upcoming resistance development of pathogenic organisms to antibiotic treatments and the toxicity of the current alternatives pose mayor disadvantages to the current treatment and prevention methods. In addition, antibiotic treatments bring along the risk of the transfer of the antibiotic substances into animal-derived products.

The current approaches therefore do not satisfy the needs of the livestock farming sector. The present invention aims to provide an alternative for the treatment and prevention of infectious hoof diseases in animals.

### SUMMARY OF THE INVENTION

The invention thereto aims to provide a composition for use in the treatment and/or prevention of infectious hoof diseases in an animal according to claim 1. More in particular, the invention provides a composition for use in the topical treatment and/or prevention of infectious hoof diseases in an animal, characterized in that said composition comprises a medium-chain fatty acid (MCFA) fraction.

The inventors have found that supplying one or more MCFA and/or MCFA derivative to bacterial pathogens causing infectious hoof diseases inhibits their further growth and that the bacteria are eventually killed by the administered MCFAs. Infectious hoof diseases are a major health challenge in the livestock farming sector affecting the productivity and the profitability of the sector. The current invention thus allows to significantly increase both the productivity and profitability of livestock farming. In addition, because infectious hoof diseases are often extremely painful and contagious, the current invention provides a composition that can make a considerable contribution to the reduction of animal suffering and the improvement of animal welfare. Finally, the current invention provides an efficient alternative for the administration of antibiotics to livestock and thus helps to prevent the upcoming spread of drug resistant pathogens.

In a second aspect, the present invention provides a method for disinfecting the hoofs of an animal according to claim 22. More in particular the invention provides a method for disinfecting the hoofs of an animal comprising the step of topically administering a composition comprising a medium-chain fatty acids (MCFA) fraction to said animal. Remarkably, regular disinfection of the hooves of the animals leads to an unexpected increase in their productivity.

Owing to the surprising bactericidal and growth inhibiting effects of MCFAs when combined with an additional carboxylic acid such as citric acid or acetic acid, the present invention provides—in a third and final aspect—a disinfectant composition according to claim 29. More in particular the invention provides a disinfectant composition comprising between 51% and 99.9% medium-chain fatty acid (MCFA) fraction and between 0.1 and 49% carboxylic acid (CA) fraction of said disinfectant composition, wherein said MCFA fraction comprises one or more MCFAs and/or derivatives thereof, and said CA fraction comprises one or more carboxylic acids.

### DETAILED DESCRIPTION OF THE INVENTION

The present invention concerns a composition for use in the treatment and/or prevention of infectious hoof diseases in an animal, wherein the composition comprises a medium-chain fatty acid (MCFA) fraction.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

The term “medium-chain fatty acid” or “MCFA” as used herein, refers to fatty acids with a medium-chain length, wherein the fatty acids may be saturated or unsaturated. MCFAs can consist of 5 to 12 carbon atoms, for example, valeric acid (C5), caproic acid (C6), caprylic acid (C8), capric acid (C10) or lauric acid (C12). The term MCFA also refers to medium-chain fatty acids that are chemically modified, and to medium-chain fatty acids that are provided with side-chains, such as, without limitation, one or more alkyl groups, preferably C1-C10 alkyl groups. As described herein, the term “medium-chain fatty acid (MCFA) derivative” refers to a fatty acid chain of which the carboxyl group is reversibly converted to a different group, preferably, but without limitation, to an amide, salt, ester or glyceride such as, for example, a mono-, di- or tri-glyceride.

The term “infectious hoof disease” as used herein refers to diseases in farm animals that affect the hoof of the animals such as, but not limited to, hairy hoof warts (papillomatous digital dermatitis), hairy heel warts, strawberry foot rot, raspberry wart, Mortellaro disease, digital dermatitis, Italian foot rot, hoof rot (interdigital phlegmon) and stable hoof rot (interdigital dermatitis).

As used herein, the term “carboxylic acid” is a moiety having a carboxyl functional group (—COOH). Unless the context clearly dictates otherwise, the term carboxylic acid includes not only the acid form, but corresponding esters and protected forms as well. The term “tricarboxylic acid” as used herein, refers to carboxylic acid whose chemical structure contains three carboxyl functional groups (—COOH).

As used herein, “therapeutically effective amount” refers to an amount of the composition which will achieve the goal of decreased microbial growth or survival in the final diluted solution. “Therapeutically effective” may also refer to improvement in disorder severity or the frequency of incidence over no treatment. The term “topical”, as used herein, refers to any composition which may be applied to the epidermis, keratin or other animal body portion.

The term “Feed Conversion Ratio” or “FCR” describes the efficiency with which the animals convert feed into the desired output. For dairy cows, for example, the output is milk, whereas in animals raised for meat (such as beef cows, pigs, chickens, and fish) the output is the flesh, that is, the body mass gained by the animal, represented either in the final mass of the animal or the mass of the dressed output.

In a first aspect, the invention provides a composition for use in the topical treatment and/or prevention of infectious hoof diseases in an animal, characterized in that said composition comprises a medium-chain fatty acid (MCFA) fraction. MCFA(s) are fatty acids with a medium-chain length, wherein the fatty acids may be saturated or unsaturated. Preferably, the composition further comprises a second carboxylic acid (CA) fraction comprising one or more carboxylic acids. More preferably the CAs in the CA fraction are selected from the group consisting of tricarboxylic acids, acetic acid and combinations thereof, most preferably from the group consisting of citric acid, acetic acid and combinations thereof. The presence of at least one of these acids in the composition leads to a surprising improvement in the efficiency of the composition. Tricarboxylic acids include, but are not limited to, citric acid, isocitric acid and aconitic acid.

In the present invention, the MCFA fraction comprises one or more MCFAs and/or derivatives thereof having a carbon chain length of between 5 and 12 carbon atoms, in other words the longest continuous chain of the MCFA consists of 5 (C5) to 12 (C12) carbon atoms. The fatty acids that can be used in this invention include both fatty acids with an even and an odd number of carbon atoms. Preferably, the MCFA fraction of the composition of the invention comprises one or more MCFAs chosen from C5 to C12 or derivatives thereof. The term MCFA also refers to medium-chain fatty acids that are chemically modified, and to medium-chain fatty acids that are provided with side-chains, such as, without limitation, one or more alkyl groups, preferably C1-C10 alkyl groups. MCFA derivatives include MCFAs of which the carboxyl group is reversibly converted to a different group such as but not limited to an amide, a salt, an ester or a glyceride such as, for example, a mono-, di- or tri-glyceride.

Infectious hoof diseases are a major health challenge in the livestock farming sector. These diseases affect both productivity and profitability of the sector, and therefore incur tremendous economic losses. The inventors have found that supplying one or more MCFA and/or MCFA derivative in the range of C5-C12, their derivatives or mixtures thereof to bacterial pathogens causing infectious hoof diseases inhibits their further growth and that the bacteria are eventually killed by the administered MCFAs. The current invention thus allows to significantly increase both the productivity and profitability of livestock farming.

Furthermore, the impact of hoof diseases on animal well-being should not be underestimated. Symptoms include decreased mobility, severe lameness, decreased milk production, body weight loss, decreased dry matter intake and decreased herd longevity. These symptoms have a detrimental effect on animal welfare and also pose a serious economic burden on producers due to lower production, increased reproductive intervals and increased culling. The current invention thus provides a composition that can make a considerable contribution to the reduction of animal suffering and the improvement of animal welfare.

In order to reduce the occurrence of hoof diseases in livestock farming systems, antibiotics are often administered to the animals. This strategy has proven largely unsuccessful in preventing or stopping hoof disease outbreaks. In addition, the problem of resistance development of pathogenic organisms and the transfer of the antibiotic substances into animal-derived products makes it very desirable to reduce the use of antibiotics in livestock farming. Therefore, the current invention provides an efficient alternative for the administration of antibiotics and thus helps to prevent the upcoming spread of drug resistant pathogens. Finally, the current invention also has the advantage that the need for administering antibiotics to the animals is decreased.

In one embodiment, the MCFA fraction of the composition comprises one or more MCFAs selected from the group of caproic acid (C6), caprylic acid (C8), capric acid (C10), lauric acid (C12) and derivatives thereof. The inventors have found that these MCFAs and their derivatives are especially efficient against hoof disease causing bacteria. In the present invention, preferably use is made of a composition comprising a mixture of specific different MCFAs, the individual MCFAs containing a different number of carbon atoms. The inventors have found that a composition wherein the MCFA comprises at least caprylic acid (C8), capric acid (C10) and lauric acid (C12) or a respective derivative is remarkably effective to treat, prevent and/or suppress infectious hoof diseases. Preferably, the MCFA fraction of the composition of the invention comprises:
Between 30 and 70% caprylic acid (C8) or a derivative thereof;
Between 15 and 55% capric acid (C10) or a derivative thereof; and
Between 1 and 35% lauric acid (C12) or a derivative thereof

based on the total weight or volume of the composition's MCFA fraction. The inventors have found that a composition comprising a mixture of MCFAs or MCFA derivatives as described above shows optimal antimicrobial properties towards the bacteria responsible for causing infectious hoof diseases.

The invention thus provides a method for treating or preventing infectious hoof diseases in an animal, comprising topically administering a therapeutically effective amount of a composition as described above to the hooves of said animal.

Due to this outstanding effectiveness on the treatment and/or prevention of infectious hoof diseases, smaller doses of the composition according to the invention need to be used to obtain the desired results on animal health and the concomitant positive effects on livestock productivity. Therefore, the current invention leads to higher revenues for the farmers while reducing the costs of disease prevention and/or treatment.

In a further embodiment, the concentration of the MCFA fraction, as described herein, amounts at least 1, 5, 10, 15, 20, 25, 30, 35, 40, 45, 50, 55, 60, 65, 70, 75, 80, 85, 90, 95, 99.9% by weight or volume of the composition. In a further embodiment, the MCFA fraction, as described herein, amounts to between 1% and 99.9%, preferably between 50% and 99.9%, more preferably between 51 and 99.9%, between 60% and 95%, based on the total weight or volume of the composition. This is to mean that the concentration of the MCFA fraction such as described herein maximally amounts to 99.9% by weight or volume of the composition.

Preferably, the total amount of CA fraction in the composition is comprised between 0.1 and 49% based on the total weight or volume of the composition, more preferably between 0.1 and 45%, even more preferably between 0.1 and 40%, between 5 and 40% based on the total weight or volume of the composition. In an embodiment the CA fraction comprises at least 1, 5, 10, 15, 20, 25, 30, 35, 40, 45, 50, 55, 60, 65, 70, 75, 80, 85, 90, 95, 99%, 99.9% preferably between 0.1 and 99.9%, more preferably between 10 and 99.9, most preferably between 50 and 99.9% citric acid and/or acetic acid, based on the total weight or volume of said composition's CA fraction. The inventors have found that within the ranges mentioned above, the efficiency of the composition for use in the prevention and/or treatment of infectious hoof diseases is further increased.

In an embodiment, the CA fraction is a mixture of citric acid and acetic acid. In an embodiment, the ratio between citric acid and acetic acid is 1:1. In another embodiment, the ratio between citric acid and acetic acid is between 1:10 and 10:1, more preferably between 1:5 and 5:1.

In an embodiment, the CA fraction is 100% citric acid. In an embodiment, the CA fraction is 100% acetic acid.

It will be appreciated that MCFA's, and possibly also acids from the CA fraction such as citric acid and acetic acid, in the present compositions will affect the pH of the composition. The pH of the composition may, however, be adjusted by the addition of acidic, basic or buffering agents. Suitable acids for use as pH adjusting agents may include, for example, sulfuric acid, sulfurous acid, sulfamic acid, hydrochloric acid, phosphoric acid, phosphorous acid, C₁-C₄ fatty acids, glycolic acid, lactic acid, acetic acid, benzoic acid, malic acid, oxalic acid, tartaric acid, succinic acid, glutaric acid, valeric acid, glycolic acid and the like. The pH may be raised, or made more alkaline, by addition of an alkaline agent such as sodium hydroxide, ammonium hydroxide, potassium hydroxide, sodium carbonate, sodium bicarbonate, monosodium acid diphosphonate or combinations thereof. Traditional acid buffering agents such as lactic acid and phosphoric acid may also be used to maintain a desired pH. The present compositions are generally acidic and, according to a further embodiment of the invention, have a pH of less than 7, preferably less than 6, more preferably less than 4, even more preferably between 1 and 5. A pH comprised within the abovementioned ranges positively contributes to the stability of the composition and therefore allows longer storage of the composition. Generally, the pH may be adjusted to any value that is desired in the intended environment of use by the addition of acid, base or buffer.

In a further embodiment of the invention, a therapeutically effective amount of said composition is administered topically to the animal. The composition for the treatment and/or prevention of infectious hoof diseases can be supplied either as a ready-to-use product or as a concentrate for dilution at the point of use. As used herein, the phrase “therapeutically effective amount” refers to an amount of the composition which will achieve the goal of decreased microbial growth or survival in the final diluted solution. “Therapeutically effective” may also refer to improvement in disorder severity or the frequency of incidence over no treatment. The term “topical”, as used herein, refers to any composition which may be applied to the epidermis, keratin or other animal body portion. The composition may be administered by pouring, squirting, flushing, sponging, or spraying it on or near the infected area. Alternatively, the animal's hoof may be soaked, submerged, or immersed in the claimed compositions to effect treatment. Accordingly, in a further embodiment the composition is administered to the animal as a spray, a foam, a gel, an ointment, a lotion, a cream, a foot bath, a foot wrap or a combination thereof.

In a further embodiment, the composition of the invention is administered to the animals as a hoof bath. A hoof bath can consist of a basin containing the composition of the invention, where the animals rest their hoofs for a determined amount of time. Alternatively, a hoof bath can also consist of a basin that stretches along a passageway wherein the basin contains the composition of the invention. In that case, the animals are allowed to walk through the passageway thereby bathing their hooves in the composition. Administration of the composition using a hoof bath has the advantage that manual manipulation of the animals' feet becomes unnecessary, thereby making the treatment and prevention of hoof diseases less cumbersome and less labor intensive. Hoof baths can be made of various materials which are suited for the passage of large animals, for example PVC or metal, and can be provided of an anti-slip cover to prevent the animals from sliding or falling. Different types of hoof baths, which are known to the skilled person, are suited for the administration of the composition. Hoof bath types range from elementary (portable) hoof baths to fully automated hoof baths which automatically fill, dose, flush and/or replenish.

In a further embodiment, the composition of the invention is administered to the animals as a hoof bath containing a liquid form of the composition. Alternatively, the composition is present in the hoof bath in foamed form. Foam has the advantage of remaining longer on the animal's skin than liquid, which extends the contact time between the hooves and/or skin of the animal and the composition for treatment, therefore promoting the antimicrobial action of the composition on the animals' hooves. A further advantage of the foam is that it is visible so it is possible to ascertain whether all the animals' feet or hooves have been carefully treated.

In a further embodiment the depth of the hoof bath is at least 1 cm, preferably at least 3 cm. This measure makes it possible to ensure that the animals' hooves come sufficiently into contact with the composition. The hoof bath's content should preferably not be deeper than 50 cm, more preferably not deeper than 30 cm.

Optionally, the feet of the animals can be cleaned before administration of the composition of the invention. Cleaning of the animals' feet can be performed manually or automatically. The latter can, for example, be performed by passing the animals through a cleaning passageway equipped with water jets or spray nozzles where water or water containing a cleaning product is applied under pressure to the animals' hooves. Of course other methods for cleaning the animals' feet can also be applied previous to treatment with the composition of the invention. Alternatively, the cleaning of the feet can be preceded by a stage where the dirt adhering to the animals' hooves is pre-softened by moistening or spraying with water or a cleaning product. Cleaning of the feet before treatment with the composition avoids extensive contamination of the hoof bath with dirt brought along on the feet of the animals. Removal of components sticking to the hooves further enlarges the direct contact between the hoof or foot skin and the composition, thereby contributing to a more efficient treatment.

As mentioned earlier, the composition for the treatment and/or prevention of infectious hoof diseases can be supplied either as a ready-to-use aqueous solution or as a concentrate composition for dilution at the point of use. Preferably dilution of the composition for use in a hoof bath is performed such that the hoof bath contains an aqueous solution comprising at least 0.5, 1, 5, 10, 15, 20, 25, 30, 35, 40, 45, 50% of the composition as described above, based on the total volume of the aqueous solution. More preferably, the dilution of the composition for use in a hoof bath is performed such that the hoof bath contains an aqueous solution comprising between 0.01% and 20%, between 0.01 and 15%, between 1 and 15%, between 1 and 10%, such as 5% of the composition as described above, based on the total volume of the aqueous solution.

The inventors have found that further the efficiency of the prevention and/or treatment of hoof diseases is improved by providing the animals with a hoof bath comprising the composition at least once every two days. Accordingly, in a further embodiment, a therapeutically effective amount of the composition is administered topically at least once every day for at least two consecutive days.

If the treatment is intended for dairy cows, as is frequently the case, the foot bath for administration of the composition of the invention can be arranged directly before and/or after the milking area. The animals therefore inevitably pass through the treatment at least two times a day, namely prior to and/or after the morning milking and prior to and/or after the evening milking. The treatment can be repeated as many days as necessary, preferably it is performed two to three consecutive days. In an embodiment, said hoofs are bathed minimal 1 time/day, by letting the ruminants cross a bath of 5 meters long.

In an embodiment the composition for topical administration to the animals may comprises one or more surfactants. Without wishing to be bound by theory, surfactants are thought to improve contact between the composition and the body surface of the animal, thus optimizing the topical administration of the composition. Surfactants are selected from the group comprising non-ionic, cationic or anionic surfactants such as, but not limited to, coconut alkylbenzyl dimethylammonium chloride, dioctyl dimethylammonium chloride, tributyl tetradecylphosphonium chloride, lauryl dimethyl amine oxide, cocoamidopropyl betaine, lauramidopropyl betaine, cocamide propyl ammonium hydroxide, alkyl benzene sulphonic acids, alkyl sulphonates, alkyl sulfates, such as sodium lauryl sulfate, alkyl aryl sulfates, alkyl aryl sulfonates, alkyl sulfonates, carboxylic acid ethers and ethoxylated and propoxylated fatty alcohols.

Infectious hoof diseases are most prominent in high density housing and intensive production dairy systems, where they pose a serious economic burden on producers due to lower production, increased reproductive intervals and increased culling. Hoofed animals which are often kept in intensive animal production facilities include sheep, goats, calves, cattle and dairy cattle. Therefore, in a further embodiment, the current composition is especially well suited for use in the topical treatment and/or prevention of infectious hoof diseases in animals selected from the group comprising sheep, goats, calves, cattle and dairy cattle.

The present composition is especially well suited for use in the treatment and/or prevention of infectious hoof diseases that can eventually result in lameness. Foot health and lameness are major issues in farming because of their common occurrence and the tremendous economic losses incurred. Prompt treatment of infectious hoof diseases can minimize the loss, improve recovery, and reduce animal suffering. Therefore, and in a further embodiment, the composition of the invention is particularly well suited for use in the treatment and/or prevention of digital dermatitis, papillomatous digital dermatitis, interdigital phlegmon, interdigital dermatitis, laminitis, white line disease, heel erosion and foot rot.

Compositions according to the present invention may optionally comprise one or more members selected from the group consisting of pH adjusting agents, wetting agents, foaming agents, dyes, viscosity control agents, preservatives, skin conditioners, coupling agents, and solvents. For example, the preservative ethylenediaminetetraacetic acid (EDTA) and its alkali salts act as chelating agents to bind metal ions that would otherwise facilitate metalloenzyme reactions that produce energy for bacterial cell replication. Other traditional preservatives may be used, for example, paraban, methyl paraban, ethyl paraban, glutaraldehyde, etc. Preservatives such as an alcohol can also be added. The alcohol, in certain embodiments, may be benzyl alcohol, a low molecular weight alcohol having a carbon number less than five, and combinations thereof.

In a second aspect, the invention provides a method for disinfecting the hoofs of animals comprising the step of topically administering to the animals a composition as described above. More in particular the method comprises the step of topically administering a composition comprising a medium-chain fatty acid (MCFA) fraction to said animals, preferably the MCFA fraction comprises one or more MCFAs or derivatives thereof having a carbon chain length of between 5 to 12 carbon atoms. In one embodiment of the method of the invention, the composition further comprises a second carboxylic acid (CA) fraction, wherein the CA fraction comprises one or more carboxylic acids. Preferably, the CAs are selected from the group consisting of tricarboxylic acids, acetic acid and combinations thereof, more preferably from the group consisting of citric acid, acetic acid and combinations thereof.

Furthermore, the total amount of CA fraction in the composition administered for disinfecting the hoofs is comprised between 0.1 and 49% based on the total weight or volume of the composition, more preferably between 0.1 and 45%, even more preferably between 0.1 and 40%, between 5 and 40% based on the total weight or volume of the composition. In an embodiment the CA fraction comprises at least 1, 5, 10, 15, 20, 25, 30, 35, 40, 45, 50, 55, 60, 65, 70, 75, 80, 85, 90, 95, 99%, 99.9% preferably between 0.1 and 99.9%, more preferably between 10 and 99.9, most preferably between 50 and 99.9% citric acid and/or acetic acid, based on the total weight or volume of said composition's CA fraction. The inventors have found that within the ranges mentioned above, the efficiency of the composition for use in the prevention and/or treatment of infectious hoof diseases is further increased.

In an embodiment, the CA fraction is a mixture of citric acid and acetic acid. In an embodiment, the ratio between citric acid and acetic acid is 1:1. In another embodiment, the ratio between citric acid and acetic acid is between 1:10 and 10:1, more preferably between 1:5 and 5:1.

In an embodiment, the CA fraction is 100% citric acid. In an embodiment, the CA fraction is 100% acetic acid.

In a further embodiment, the concentration of the MCFA fraction, as described herein, amounts at least 1, 5, 10, 15, 20, 25, 30, 35, 40, 45, 50, 55, 60, 65, 70, 75, 80, 85, 90, 95, 99.9% by weight or volume of the composition. In a further embodiment, the MCFA fraction, as described herein, amounts to between 1% and 99.9%, preferably between 50% and 99.9%, more preferably between 51 and 99.9%, between 60% and 95%, based on the total weight or volume of the composition. This is to mean that the concentration of the MCFA fraction such as described herein maximally amounts to 99.9% by weight or volume of the composition.

The inventors have found that supplying one or more MCFA and/or MCFA derivative in the range of C5-C12, their derivatives or mixtures thereof to inhibits their further growth and that the bacteria are eventually killed by the administered MCFAs. Topical administration of the composition of the invention as described above to the hooves of the animals, thus leads to efficient disinfection of the hooves of those animals. In one embodiment, disinfection of the hooves according to the method of the invention is performed at least once every two days. Regular disinfection of the hooves of the animal surprisingly results in an unexpected increase in their productivity. This positive effect can be measured, for example, based on the animals' growth or based on the nutritional value of the animals and their derived products. Evidently, other methods to measure the productivity of the animals, which are known to the person skilled in the art can also be used in combination with the invention. For instance, growth can be measured at the end of the growing period as the animal's weight or the animal's size. Alternatively, the animal's growth can be measured over a determined period of time resulting in the growth rate of the animal over time. The productivity can furthermore also be measured, for example, as the milk production capacity of the animal, in the case of dairy cattle.

Farmers often evaluate the productivity of animals based on the measurement of their “feed conversion ratio” or “FCR”. The FCR describes the efficiency with which the animals convert feed into the desired output. For animals raised for meat (such as beef cows, pigs, chickens and fish) the output is the flesh, that is, the body mass gained by the animal, represented either in the final mass of the animal or the mass of the dressed output. For animals raised for dairy production, the output is the milk production. The inventors have found that efficient disinfection of the hooves of an animal leads to increased milk production, increased immunity and/or improved meat production.

The inventors have found that due to its negative effect on bacterial growth and viability, the composition described above is a surprisingly efficient disinfectant composition. Therefore, and in a final aspect, the invention provides a disinfectant composition as described in one of the embodiments above.

The invention is further described by the following non-limiting examples which further illustrate the invention, and are not intended to, nor should they be interpreted to, limit the scope of the invention.

### EXAMPLES

### Example 1: Disinfectant Activity of the Composition According to an Embodiment of the Invention

Dichelobacter nodosus, formerly Bacteroides nodosus, is a Gram-negative, obligate anaerobe of the family Cardiobacteriaceae. It has polar fimbriae and is the causative agent of ovine foot rot as well as interdigital dermatitis.

The “minimum inhibitory concentration” or “MIC” of a composition comprising 90% MCFA fraction wherein 50% was caprylic acid, 30% was capric acid and 20% was lauric acid, and 10% CA fraction comprising citric acid, was determined. As used herein, MIC refers to the lowest concentration of a compound or composition which prevents measurable growth of a bacterium.

An equal CFU of Dichelobacter nodosus was plated on suitable growth medium containing 0% (control), 0.5%, 1.5%, 2.5%, 5% or 50% dilution of the composition described above.

D. nodosus growth inhibition was not observed under control conditions, however a 0.5% dilution of the composition was already enough to inhibit growth of the bacterium. Therefore, the MIC of the composition is 0.5%.

### Example: Disinfecting Hoofs of Dairy Cows Activity of the Composition According to an Embodiment of the Invention

A hoof bath was provided comprising 5% of a disinfectant composition, comprising 95% of MCFA and 5% of citric acid. Hoofs of ruminants were bathed minimal 1 time per day, by letting the ruminants cross a hoof bath of 5 meters long. Hoof health was inspected after at least one week treatment and was considered good. Results were maintained over time.

Similar effects were obtained by use of a composition with lower MCFA concentration and higher citric acid, as well as with a combination of citric acid and acetic acid.

It is supposed that the present invention is not restricted to any form of realization described previously and that some modifications can be added to the presented example without reappraisal of the appended claims.

1. A composition for use in the topical treatment and/or prevention of infectious hoof diseases in an animal, characterized in that said composition comprises a medium-chain fatty acid (MCFA) fraction.

2. The composition for use according to claim 1, characterized in that said composition further comprises a second carboxylic acid (CA) fraction comprising of one or more carboxylic acids.

3. The composition for use according to claim 2, characterized in that said carboxylic acids are selected from the group consisting of tricarboxylic acids, acetic acid and combinations thereof.

4. The composition for use according to claim 2, characterized in that said carboxylic acids are selected from the group consisting of citric acid, acetic acid and combinations thereof.

5. The composition for use according to claim 2, characterized in that said CA fraction comprises between 0.1 and 99.9% citric acid and/or acetic acid, of said composition's CA fraction.

6. The composition for use according to claim 1, characterized in that said MCFA fraction comprises one or more MCFAs and/or derivatives thereof having a carbon chain length of between 5 and 12 carbon atoms.

7. The composition for use according to claim 1, characterized in that said MCFAs are selected from the group of caproic acid (C6), caprylic acid (C8), capric acid (C10), lauric acid (C12) and derivatives thereof.

8. The composition for use according to any one of the previous claims claim 1, characterized in that said MCFA fraction comprises at least caprylic acid (C8), capric acid (C10) and lauric acid (C12) or a derivative thereof.

9. The composition for use according to claim 8, characterized in that said MCFA fraction comprises
between 30% and 70% caprylic acid (C8) or a derivative thereof;
between 15% and 55% capric acid (C10) or a derivative thereof; and
between 1% and 35% lauric acid (C12) or a derivative thereof;
of said composition's MCFA fraction.

10. The composition for use according to claim 1, characterized in that the total amount of said MCFA fraction in said composition is comprised between 51% and 99.9% of the composition.

11. The composition for use according to claim 2, characterized in that the total amount of CA fraction in the composition is comprised between 0.1 and 49% of the composition.

12. The composition for use according to claim 1, characterized in that said composition has a pH of below 7.

13. The composition for use according to claims claim 1, characterized in that said composition comprises one or more surfactants.

14. The composition for use according to any of the previous claims claim 1, characterized in that a therapeutically effective amount of said composition is administered topically to said animal.

15. The composition for use according to claim 1, characterized in that said composition is administered as a spray, a foam, a gel, an ointment, a lotion, a cream, a foot bath, a foot wrap or a combination thereof.

16. The composition for use according to claim 1, characterized in that said composition is administered as a hoof bath.

17. The composition for use according to claim 17, characterized in that an aqueous solution comprising 0.01 to 20% of the composition according to any one of the previous claims is provided in said hoof bath, based on the total volume of said aqueous solution.

18. The composition for use according to claim 1, characterized in that a therapeutically effective amount of said composition is administered topically at least once every day for at least two consecutive days.

19. The composition for use according to claim 1, characterized in that said animal is selected from the group comprising sheep, pigs, horses, goats, calves, cattle and dairy cattle.

20. The composition for use according to claim 1, characterized in that said infectious hoof disease is selected from the group comprising digital dermatitis, papillomatous digital dermatitis, interdigital phlegmon, interdigital dermatitis, laminitis, white line disease, heel erosion and foot rot.

21. A method for disinfecting the hoofs of an animal comprising the step of topically administering a composition comprising a medium-chain fatty acid (MCFA) fraction to said animal, wherein said MCFA fraction comprises one or more MCFAs and/or derivatives thereof.

22. The method according to previous claim 21, characterized in that said composition comprises a second carboxylic acid (CA) fraction, wherein the CA fraction comprises one or more carboxylic acids.

23. The method according to previous claim 23, characterized in that said carboxylic acids are selected from the group consisting of tricarboxylic acids, acetic acid and combinations thereof.

24. The method according to claim 23, characterized in that said CA fraction comprises between 0.1 and 99.9% citric acid and/or acetic acid of said composition's CA fraction.

25. The method according to claim 22, characterized in that the total amount of said MCFA fraction in said composition is comprised between 51% and 99.9% of the composition.

26. The method according to claim 22, characterized in that said composition is administered at least once every day for at least two consecutive days.

27. The method according to claim 22, characterized in that said animals are selected from the group of goats, sheep, calves, cattle and dairy cattle.

28. The method according to claim 22, wherein said disinfection occurs by providing a liquid solution comprising said composition suitable for being used as a hoof bath, and wherein said concentration of said composition in said liquid is between 0.01% and 20%.

29. A disinfectant composition comprising between 51% and 99.9% medium-chain fatty acid (MCFA) fraction and between 0.1 and 49% carboxylic acid (CA) fraction of said disinfectant composition, wherein said MCFA fraction comprises one or more MCFAs and/or derivatives thereof, and said CA fraction comprises one or more carboxylic acids.

30. The disinfectant composition according to claim 29, characterized in that said MCFA fraction comprises
between 30 and 70% caprylic acid (C8) or a derivative thereof;
between 15 and 55% capric acid (C10) or a derivative thereof; and
between 1 and 35% lauric acid (C12) or a derivative thereof;
of said composition's MCFA fraction.

31. The disinfectant composition according to claim 29, characterized in that said CA fraction comprises between 0.1 and 99.9% citric acid and/or acetic acid, of said composition's CA fraction.
